(19) United States
(12) Patent Application Publication
(10) Pub. No.: US 2014/0330067 A1
(43) Pub. Date: Nov. 6, 2014

(54) Device for extracorporeal magnetic innervation

(71) Applicant: Michael Jordan, Munchen (DE)

(72) Inventor: Michael Jordan, Munchen (DE)

(21) Appl. No.: 14/004,814

(22) PCT Filed: Mar. 16, 2012
(86) PCT No.: PCT/EP12/54717

§ 371 Date: Oct. 21, 2013

(30) Foreign Application Priority Data
Mar. 17, 2011 (DE) 10-2011-014-291.6

(51) Int. Cl. A61N 2/02

(52) U.S. Cl. CPC A61N 2/02
USPC 600/13

(57) ABSTRACT

A device is for extracorporeal magnetic innervation. The device allows a non-invasive, non-operative treatment using magnetic fields. In particular, the device is suitable for nerve stimulation by means of the magnetic fields which are generated, thereby resulting in the contraction of a muscle.

### FIELD OF THE INVENTION

The invention relates to extracorporeal magnetic innervation (ExMI) which is an extracorporeal magnetic stimulation. In particular, the invention relates to an incontinence treatment device for the treatment and cure of incontinence, to a chair having an incontinence treatment device and to a wheelchair having an incontinence treatment device. The invention also relates to a device for slowing down muscle regression, which device stimulates nerves in a muscle, thereby slowing down the regression of the muscle, to a hand-held unit for slowing down muscle regression, and to a couch for slowing down muscle regression. Furthermore, the invention relates to a body fat break-down device which stimulates nerves in a muscle such that body fat is broken down. The invention further relates to a sports-medicine muscle development accelerating device which stimulates nerves in a muscle, thereby assisting muscle development, and to a couch for sports-medicine muscle development acceleration. The invention also relates to a stroke rehabilitation device which stimulates nerves in a muscle, thereby assisting rehabilitation following a stroke, and to a couch for stroke rehabilitation. Furthermore, the invention relates to a pelvic floor muscle rehabilitation device for postnatal use, which device stimulates nerves in a muscle, thereby assisting rehabilitation and redevelopment of the pelvic floor muscles following childbirth. The invention also relates to a blood flow stimulating device which stimulates nerves in a muscle, thereby stimulating the flow of blood through the tissue, and to a couch for stimulating blood flow.

### TECHNICAL BACKGROUND OF THE INVENTION

The use of magnetic fields for triggering action potentials in cells is known. Here, a stimulus is applied, for example in the form of a magnetic field, to the surface of a patient's skin or the stimulus slightly penetrates the patient's skin, for example by a few millimetres.

In addition, it is also known to trigger a corresponding pulse in a patient by applying electrical pulses to the patient's skin.

### SUMMARY OF THE INVENTION

It can be considered as an object of the invention to provide a device for a non-operative, non-invasive treatment of a person, the device being characterised in that it produces contractions of the muscles due to a controlled stimulation of nerves.

Provided according to the features of the independent claims are an incontinence treatment device, an incontinence treatment chair, an incontinence treatment wheelchair, a device for slowing down muscle regression, a hand-held unit for slowing down muscle regression, a couch for slowing down muscle regression, a body fat break-down device, a sports-medicine muscle development accelerating device, a couch for sports-medicine muscle development acceleration, a stroke rehabilitation device, a couch for stroke rehabilitation, a pelvic floor muscle rehabilitation device for postnatal use, a blood flow stimulating device, and a couch for stimulating blood flow. Developments of the invention are provided in the dependent claims.

### Incontinence Treatment, Incontinence Prevention and Continence Training

According to a first aspect of the invention, an incontinence treatment device is provided which comprises a coil, an energy supply unit and a control unit.

In this respect, the coil is configured such that it can generate a magnetic field which can stimulate nerves in a muscle of a patient who is within the active region of the coil.

The energy supply unit provides an energy supply for the coil. In particular, the energy supply unit can be configured such that it can provide high currents, for example currents of up to 3000 A or more. The energy can be supplied in the manner of pulses with an alternating pulse duration and alternating interruptions in the power supply between the pulses.

Of course, the currents provided by the energy supply unit can also exceed the above-mentioned value of 3000 A, if this should be necessary due to external parameters. For example, an amperage of up to 5000 A or more may be necessary, which is then also provided accordingly by the energy supply unit.

Pulse duration and interruptions between the current pulses as well as the amperages can be adapted, for example, to the objective of the treatment, subject to the purpose.

The control unit is configured such that it can control the energy supply to the coil by the energy supply unit. In particular, the energy supply is controlled by the control unit such that the magnetic fields generated by the coil are capable of treating and curing accompanying symptoms of incontinence.

The coil is a device comprising a plurality of windings of a wire. The windings can be wound around a magnetic, a non-magnetic or a ferromagnetic core.

The wire used for the coil can be in particular copper wire. The coil can have in particular 10 windings. Depending on the geometric shape of the coil core, said core can have in particular a diameter or an edge length in the transverse direction of 15 to 28 cm. In the longitudinal direction, the coil core can be of any extent.

Of course, this does not rule out using another wire, more or fewer windings or a core with other dimensions. In particular, when configuring the coil and coil core, it is crucial that the generated magnetic field has a penetration depth into the patient's body of at least approximately 7 to 8 cm.

The wire of the coil can have a variable cross section. In particular, the cross section of the wire can be adapted to an amperage which is supplied by the energy supply unit. In this respect, the greater the amperage, the larger the cross section must be. The cross-sectional area of the wire can be between, for example 0.1 mm² and 80 mm², but in the case of greater amperages, it can also exceed this value, for example it can be up to 150 mm².

The amperage for operating the coil and for generating a magnetic field is provided subject to external parameters, in particular predetermined by the person to be treated. These parameters can be size, weight, waist circumference, pelvic circumference and shoulder circumference. Of course, other parameters can also be used, for example the length of individual bones, for example the length of the femur.

In particular, the nature of the disorder to be treated and the treatment objective to be achieved are very important for the setting of the amperage, the pulse duration of the current and the length of the interruptions of the current pulses.

Designated as the active region of the coil is the space surrounding the coil in which the magnetic field generated by the coil is strongest. In this active region, the magnetic field is capable of stimulating nerve cells in a muscle such that the corresponding muscle cells contract. In particular, it is thereby possible to cause local muscle contractions, without the entire muscle contracting.

The magnetic field generated by the coil can penetrate inside a patient's body by several centimetres, in particular by at least 7 cm. In this respect, the muscles are activated by the stimulation of all branches of the pudendal and splanchnic nerves. The magnetic fields penetrate clothing, bones and soft tissue in order to stimulate the nerve cells. The electrical potential generated by the magnetic field produces an ion flow or an eddy current in the soft tissue. This ion flow results in a depolarisation of the resting motor neurons. Upon reaching a polarisation threshold, an action potential is initiated for the corresponding neuron, which potential spreads in a natural manner along the axon via the normal Na+ and K+ ion flow. When these pulses reach the motor endplates, the muscles react by contraction according to the frequency of the output pulses of the magnetic field generated by the coil. The muscles contract and relax with each pulse, as long as the frequency of the output pulses does not exceed the contraction and relaxation capability of the muscles. As a result, the muscles contract constantly.

In particular, the invention in all embodiments described above and in the following can also exhibit its effect in a patient's body through dressings, plaster casts and clothing. Due to the characteristics of the magnetic field, the effect can take place through any item resting against the body, provided that this item does not have a magnetic shielding action.

The pulse duration of the power supply of the coil and the interruption in the power supply between the pulses (denoted hereafter as interruption time) can be selected such that the natural contractility of the muscle is not exceeded. In particular, a different pulse frequency can be selected subject to the treatment objective.

For the treatment of incontinence, it is provided to strengthen the muscle endurance, the muscle volume and the muscle power of the sphincter. Subject to the diagnosis, these three objectives can differ in significance. Thus, in the case of stress incontinence, it is possible to start earlier with the development of muscle power during treatment than in the case of mixed incontinence.

A low pulse frequency of up to 10 Hz is provided to improve the muscle endurance. The muscle volume is improved by a pulse frequency in the medium range between 20 Hz and 30 Hz, in particular at 23 Hz. An improvement in muscle power is achieved by a pulse frequency close to the natural contractility of human musculature, in particular at 50 Hz.

Of course, the pulse frequency can vary subject to the individual requirements of the patient and, according to an embodiment of the invention, these individual requirements can be recorded so that pulse frequency and/or amperage can be adjusted automatically.

In particular, a pulse frequency which alternates regularly or irregularly but also randomly can improve the success of the treatment in an advanced stage of treatment and can contribute to the long-term maintenance thereof.

A session can be of a variable duration, depending on the patient, the diagnosis and the desired treatment objective. In particular, a session can last 20 minutes and can be divided into two 10 minute portions, there being a pause of two minutes between the first portion and the second portion.

The objective of the treatment is achieved when the muscle rehabilitation or the desired muscle build up has been attained. Achievement of the objective is to be established individually for each patient. The objective can be achieved in particular by 40 ten minute sessions or also by 20 sessions of twenty minutes each.

In the context of this invention, in particular uncontrolled leakage of urine and a too frequent urge to urinate are understood as accompanying symptoms of incontinence which are to be treated, as are all other medical accompanying symptoms associated with this disorder.

According to another embodiment of the invention, the device can also be configured for incontinence prevention and for continence training. In this embodiment, in particular the pulse frequency can be adapted to the treatment objective and it possibly differs from the pulse frequency for the treatment of incontinence.

The control unit which controls the energy supply of the coil by the energy supply unit can be set manually and automatically. In particular, the control unit predetermines the pulse frequency, the on-time, the off-time and the energy or amperage for the operation of the coil.

The on-time is the time in which the coil is supplied with energy and generates a corresponding magnetic field with the set pulse frequency.

The off-time is the time in which the coil is not supplied with energy.

In other words, the on-time indicates a contraction time of the muscles, thus a stress time, and the off-time indicates the rest time of the muscles.

The energy or amperage with which the coil is operated directly influences the intensity and the form of the magnetic field generated by the coil. This means that the active region of the magnetic field is fixed by the setting of the energy or amperage. In particular, a penetration depth of the magnetic field into a patient's body can be predetermined hereby, which is necessary due to the varying individual nature of the human body. The active region is to be set such that the muscle to be treated is located within the active region of the magnetic field.

The control unit can also be adjusted in an automated manner, in particular in that after the disorder and further necessary patient data has been established, the set values for the control unit are recorded on a patient's card.

The patient's card can be a computer-readable storage medium which contains the values for the pulse frequency, the on-time, the off-time and the energy or amperage.

The computer-readable storage medium can be, for example, a USB stick, a flash card, a floppy disc, a CD ROM or any other computer-readable storage medium. In particular, it can be a chip card.

The core of the coil can assume any shape. It can be an oblong or curved core, for example in the shape of a semi-circle. However, a plurality of cores can also be used. In this respect, a single coil can wind round this plurality of cores, but it is also possible for an individual coil to wind round each individual core of the plurality of cores.

It is pointed out that in the context of this invention, a device having a plurality of cores is also denoted as a coil, both in the configuration in which one coil winds round a plurality of cores and in the configuration in which an individual coil winds round each core.

According to an embodiment of the invention, the incontinence treatment device also comprises a guide device and an actuator. The guide device is configured such that the coil can be moved along the guide device, so that the position of the coil is changed. The actuator is configured such that it can undertake the positioning of the coil on the guide device.

The guide device can be, for example, a rail. In this respect, the guide device is configured such that it allows a positioning and fixing of a coil attached thereto.

The guide device can also comprise a plurality of guide elements, for example rails. It can thereby be possible, for example, to move the coil in three spatial directions which are vertical to one another in each case. It is thereby possible to predetermine a longitudinal position and a transverse position as well as a vertical position. The penetration depth of the magnetic field can be influenced in particular by the vertical positioning.

Particularly in the case in which the guide device comprises a plurality of guide elements, by changing the position of one guide element relative to the other guide elements, it is possible to produce not only a change in the position of the coil, but also a change in the flux direction of the magnetic field lines in space.

The guide elements of the guide device can be configured such that the coil can be moved in a continuous manner. However, the guide elements can also be configured such that the coil can be moved in predetermined steps. In this respect, the steps can be provided by elevations or notches in the guide elements.

It is possible to use as the actuator any component which is capable of producing a movement by the supply of energy, for example electrical or chemical energy. This component can be an electric motor, for example. However, it can also be a hydraulic or pneumatic element. The actuator can be assisted by an energy-storing element, for example a spring. However, the actuator can also be a crank, for example, which is turned manually by an operator to thus change the position of a coil, for example via a rotating threaded rod.

The invention is not only suitable for the treatment of incontinence, but it also allows a non-operative, non-invasive treatment in the entire region of the pelvic floor. This includes, for example, the treatment of urinary and faecal incontinence, incontinence following radical prostatectomy, pelvic pain, erectile dysfunction and other sexual dysfunctions, haemorrhoids and vaginal tonus.

Depending on the diagnosis and on the treatment objective, the invention can be used to regain and improve the strength and endurance of a muscle.

During the treatment of incontinence, the invention makes it possible to restore bladder control.

The table which is shown later on illustrates exemplary areas of use of the invention with associated settings of the control unit. It is to be stressed in particular that the areas of use mentioned in the following are not to be considered as a conclusive list and the mentioned exemplary settings are merely indications which can vary depending on the patient. The actual values of the parameters of the control unit can only be established following a diagnosis of the disorder.

The parameters for setting the control unit form a patient-based treatment program. This treatment program can be stored on a computer-readable medium, so that at the start of the session, the treatment program is read out from the computer-readable storage medium by the control unit and the control unit is adjusted accordingly.

A chip card which can be read out by the control unit via an integrated data card reader is particularly recommended as a computer-readable storage medium.

The automation of the settings of the parameters of the control unit reduces the configuration time and setting errors are prevented.

Of course, the invention can be used both for men and for women.

A further embodiment of the invention describes an incontinence treatment chair. The incontinence treatment chair has a seating surface with an incontinence treatment device as described above and in the following. In this respect, the incontinence treatment device can be arranged in particular under the seating surface.

For the treatment by the invention, a patient sits on the seating surface of the incontinence treatment chair. The patient can change his sitting position so that the muscle to be treated is brought into the active region of the magnetic field generated by the coil. However, the patient can also adopt a constant sitting position which can be repeated, the sitting position being indicated by at least one reference point. A reference point of this type can be, for example, a backrest of the incontinence treatment chair. The backrest ensures that the distance of a part of the patient's body is always at the same distance from the backrest when the patient's back contacts the backrest.

To prevent a sideways movement of the patient relative to the backrest, a reference point, vertical to the backrest, can be attached. This can be, for example, a device similar to an armrest. During a sideways movement of the patient relative to the backrest such that the lateral reference point is contacted by the patient's body, for example by the patient's hip, it is ensured that the muscle to be treated is always in the same position even in a lateral direction.

The distance of the muscle to be treated from the seating surface is predetermined alone by the body measurements of the patient during any sideways or forwards or backwards movement of the patient on the chair.

The spatial positioning of the muscle to be treated is known thereby. Due to this spatial positioning, the position of the coil can be adjusted by the control unit so that the muscle to be treated is within the active region of the magnetic field of the coil.

A change in the patient's body dimensions affects the spatial positioning of the muscle to be treated. The position of the coil has to be adapted accordingly.

To prevent a reduction of the penetration depth of the active region of the magnetic field generated by the coil into the patient's body, the seating surface of the incontinence treatment chair can be configured such that it has a relatively smaller material thickness in predetermined areas. Furthermore, the reference points for a sideways and forwards and backwards positioning of the patient on the incontinence treatment chair can be mounted in a movable manner, so that the patient can always be positioned on the chair such that a muscle to be treated is located above a region of the seating surface which has a relatively small thickness, so that achieving the maximum penetration depth of the magnetic field into the patient's body can always be ensured.

A further embodiment of the invention provides an incontinence treatment wheelchair. The incontinence treatment wheelchair has a seating surface and an incontinence treatment device as described above and in the following. In this respect, the incontinence treatment device is located under the seating surface of the wheelchair.

An incontinence treatment wheelchair can facilitate in particular the treatment of patients who are dependent on a wheelchair.

The incontinence treatment device can be attached in a modular manner to the wheelchair. This makes it possible to attach the incontinence treatment device to the wheelchair prior to treatment and to remove it at the end of treatment. This facilitates the treatment of patients who rely on a wheelchair, because these patients do not have to be relocated for their treatment.

The arrangement, described above, comprising a coil, an energy supply unit and a control unit can also be used for the devices described in the following.

### Slowing Down or Prevention of Muscle Regression

A further aspect of the invention provides a device for slowing down muscle regression. The device for slowing down muscle regression comprises a coil described above and in the following, an energy supply unit described above and in the following and a control unit described above and in the following.

According to another embodiment of the invention, the device can also be configured to prevent muscle regression or to build up muscle. In this respect, in particular the pulse frequency can be adapted to the treatment objective.

In this respect, the coil is configured such that a magnetic field is generated which stimulates the nerves in a muscle of a patient located within an active region of the coil. The energy supply unit provides a supply of energy for the coil, the energy supplied to the coil by the energy supply unit being controlled by the control unit.

In this respect, the energy supply is controlled by the control unit in particular so that the magnetic fields generated by the coil are capable of stimulating nerves in a muscle of a patient located within an active region of the coil, so that a muscle activity resulting from the nerve stimulation slows down muscle regression.

Thus, the invention is particularly suitable for post-operative use in sports men and women, for whom a few days during which the muscles are not stressed have a noticeably negative effect.

The negative consequences of sporting inactivity are loss of strength endurance, maximum strength and volume of a muscle.

These negative consequences are to be prevented or are to be minimised as far as possible.

The coil, the energy supply unit and the control unit have characteristics as described above.

For example, it is appropriate to use the invention to maintain the thigh muscles following an operation on a patient's knee. The muscles which are substantially maintained or, in an ideal case, are entirely maintained contribute in a positive manner to a relatively rapid recovery and rehabilitation following the operation. Furthermore, the muscle characteristics which are lost do not have to be regained in a protracted build-up phase. This advantage becomes particularly apparent in that the build-up of the muscles requires a multiple of the inactivity time of the muscles. Thus, a sports person can return relatively quickly to his daily competitive routine.

Of course, the invention can also be used to maintain muscle in the case of coma patients or in the case of patients who are unable to move.

To slow down muscle regression, in particular pulse frequencies for the coil are suitable within the medium and high frequency ranges (from approximately 20 Hz up to the maximum natural contractility of a muscle, for example 50 Hz). With frequencies in the medium range (20 Hz to 25 Hz), a muscle volume is improved or maintained and with frequencies in the high range (approximately 50 Hz, at most up to the natural contractility of a muscle), the muscle strength is maintained or preserved.

Of course, depending on the patient's constitution, pulse frequencies in the low range (2 Hz to 15 Hz) can also be used to improve or maintain the muscle endurance of a treated muscle.

Particularly in the advanced stage of a treatment, the pulse frequency can change regularly, irregularly or randomly.

An embodiment of the invention describes a hand-held unit for slowing down muscle regression. In this respect, the hand-held unit comprises a device for slowing down muscle regression according to an aspect of the invention.

The hand-held unit can be configured such that in a mobile part, it only has the coil of the device for slowing down muscle regression. The energy supply unit and the control unit can be connected to the hand-held unit so that the coil can be supplied with energy. This construction makes it possible to provide a lightweight hand-held device.

A further embodiment of the invention describes a couch for slowing down muscle regression. In this respect, the couch has a lying surface, a device for slowing down muscle regression according to an aspect of the invention, a guide device and an actuator.

The guide device is configured such that the position of a coil along the guide device can be changed. The actuator is provided to carry out the positioning of the coil on the guide device.

The couch can of course be provided with a plurality of guide devices or guide elements. In this respect, at least one coil can be attached to each guide element or to each guide device.

The guide devices can be attached under the lying surface, on the side of the lying surface or above the lying surface. However, the guide devices can also be configured such that they surround the lying surface in the form of a circle. In this respect, the circular guide device can be attached such that the surface which it describes is parallel to a longitudinal direction or is parallel to a transverse direction of the couch.

The guide devices can be arranged under the lying surface such that they basically reflect the shape of important muscles in the human body. This allows coils which move along this guide device to be moved along an entire muscle. A magnetic field generated by a coil of this type is thereby able to make an entire muscle contract in successive different portions.

Of course, the guide devices can also be arranged in a movable manner so that patients having different body dimensions can be mapped in the form of the guide devices.

Guide devices which are arranged on the side of the lying surface or above the lying surface are particularly suitable for the stimulation of nerve cells and muscles which are located on the side of a patient's body.

To spare a patient a possibly unpleasant and uncomfortable abdominal position on the couch, a guide device for a coil can be arranged such that a coil can move above the lying surface and above a patient who is lying on his back, and muscles, for example in the chest or abdominal region of a patient can be stimulated thereby.

A guide device which runs circularly around the lying surface allows the positioning of a coil so that muscles in the entire region of a patient's body can be stimulated.

A guide device which runs circularly around the lying surface is particularly suited to travelling over a patient's body in the longitudinal direction, in which case the guide device can be configured such that it adapts to the contour of the patient's body as it moves in a longitudinal direction of the patient. This can ensure a uniform penetration depth of the magnetic field of the coil into the patient's body.

Equally suitable for travelling over the contour of a patient's body is a guide device in the form of a swivel arm, on the movable end of which is attached a coil for generating a magnetic field. A swivel arm affords the particular advantage that the movable end can be freely spatially positioned.

A couch as described above and in the following can be in particular a hospital bed.

The circular guide devices for the coil can also be configured such that a circular guide device of this type can travel over extremities such as the arms and legs of a patient.

### Break Down of Body Fat

A further aspect of the invention describes a body fat break-down device. The body fat break-down device comprises a coil described above and in the following, an energy supply unit described above and in the following, and a control unit described above and in the following.

In this respect, the coil is configured such that a magnetic field is generated which stimulates the nerves in a muscle of a patient who is within the active region of the coil. The energy supply unit provides the energy supply for the coil. The control unit is arranged to control the energy supply of the coil by the energy supply unit.

In this respect, the energy supply is controlled by the control unit so that the magnetic fields generated by the coil are capable of stimulating the nerves in a muscle of a patient who is within the active region of the coil, so that body fat is broken down.

The use of a body fat break-down device as described above and in the following can naturally be configured both as a hand-held unit and as a couch.

A body fat break-down device is particularly suitable for use in patients who, due to physical deficiencies or other afflictions, for example, are restricted in carrying out physical activities. Physical activity is an essential part in assisting and promoting metabolism. If a physical activity is no longer possible or is only possible to a limited extent, an appropriate muscle activity can be produced by the invention.

In order to map as far as possible a holistic stressing of the muscles, a pulse frequency which changes regularly, irregularly or randomly is possible in particular for the coil generating the magnetic field. With the alternation of the pulse frequency from, for example 2 Hz up to the maximum contractility of the muscles, for example 50 Hz, the muscle endurance, muscle volume and muscle power is stressed and the muscle is stimulated into consuming a great amount of energy, thereby making it possible for body fat to be broken down.

The coil, the energy supply device and the control unit have characteristics as described above.

### Build-up of Muscle

A further aspect of the invention provides a sports-medicine muscle development accelerating device which comprises a coil described above and in the following, an energy supply unit described above and in the following, and a control unit described above and in the following.

In this respect, the coil is configured such that it generates a magnetic field which stimulates the nerves in a muscle of a patient who is within the active region of the coil. The energy supply unit provides an energy supply for the coil. The control unit is arranged such that it controls the energy supply of the coil by the energy supply unit.

In this respect, the energy supply is controlled by the control unit so that the magnetic fields generated by the coil are capable of stimulating the nerves in a muscle of a patient who is within the active region of the coil, so that due to the muscle contractions resulting therefrom, a build-up of muscle is assisted.

The information provided above applies to the coil, to the energy supply unit and to the control unit.

According to the purpose of the muscle development accelerating device, it is particularly appropriate to operate the coil with a pulse frequency in the medium and high ranges (approximately 20 Hz up to the maximum contractility of a muscle, approximately 50 Hz).

The muscle development accelerating device can be used to assist the build-up of muscle as part of a training programme. However, it can also be used in conventional muscle development exercises. For example, a sports exercise appliance for building up muscle can be provided with a muscle development accelerating device.

However, the muscle development accelerating device can also be used on its own to build up a muscle. In particular, the duration of a treatment session and the number of daily sessions are decisive factors here.

The pulse frequency of the coil and the duration and number of treatment sessions have to be coordinated during the treatment sessions. In this respect, a higher pulse frequency of the coil corresponds to a greater contraction intensity of the muscle and thereby ensures a greater fatigue of the muscles. To avoid over-stressing the muscles, the number of treatment sessions is to be reduced accordingly.

An embodiment of the invention provides a couch for sports-medicine muscle development acceleration, the couch having a lying surface, a muscle development accelerating device according to an aspect of the invention, a guide device and an actuator.

The actuator is provided to position the coil on the guide device. The position of the coil can be changed along the guide device. Furthermore, the control unit can control the actuator to position the coil.

The information provided above applies to the actuator, to the guide device and to the arrangement of the muscle development accelerating device or to the coil on the guide device.

### Stroke Rehabilitation

A further aspect of the invention provides a stroke rehabilitation device which comprises a coil described above and in the following, an energy supply unit described above and in the following, and a control unit described above and in the following.

In this respect, the coil is configured such that a magnetic field is generated which stimulates the nerves in a muscle of a patient who is within the active region of the coil. The energy supply unit provides the energy supply for the coil and the control unit is arranged to control the energy supply of the coil by the energy supply unit.

In this respect, the energy supply is controlled by the control unit so that the magnetic fields generated by the coil are capable of stimulating the nerves in a muscle of a patient who is within the active region of the coil so that, due to pulses resulting therefrom, in a neural pathway, rehabilitation following a stroke is assisted and promoted.

The information provided above applies to the coil, to the energy supply unit and to the control unit.

The nerve cells are polarised in the active region of the magnetic field generated by the coil. The signal produced thereby in a neural pathway propagates both in the direction of the muscles and in the direction of the brain. Thus, a muscle receives a movement pulse and the brain receives a pulse which corresponds to the movement of the stimulated muscle.

After a stroke, it can happen that areas of the brain responsible for controlling specific muscle groups can no longer perform their intended tasks. These tasks may be taken over by other areas of the brain. This new linking procedure of the muscles with the corresponding area of the brain can be assisted by the invention in that the brain receives a signal which it can assign to an actually matching muscle contraction.

An embodiment of the invention provides a couch for stroke rehabilitation which comprises a lying surface, a stroke rehabilitation device according to an aspect of the invention, a guide device and an actuator.

The information provided above applies to the actuator, to the guide device and to the lying surface.

The stroke rehabilitation device can naturally also be used in the case of stroke patients to maintain and to build up muscle.

### Pelvic Floor Muscles

A further aspect of the invention provides a pelvic floor muscle rehabilitation device for postnatal use which comprises a coil described above and in the following, an energy supply unit described above and in the following, and a control unit described above and in the following.

In this respect, the coil is configured such that it generates a magnetic field which stimulates the nerves in a muscle of a patient who is within the active region of the coil. The energy supply unit is configured to provide the energy supply for the coil. The control unit is arranged to control the energy supply of the coil by the energy supply unit.

In this respect, the energy supply is controlled by the control unit so that the magnetic fields generated by the coil are capable of stimulating the nerves in a muscle of a patient who is within the active region of the coil, so that due to the muscle contractions resulting therefrom, rehabilitation and redevelopment of the pelvic floor muscles following childbirth are assisted.

Childbirth presents a major strain on a woman's pelvic floor. It may result in tissue tears and damage to the pelvic floor muscles and to the back of the woman who is giving birth. Serious long-term effects can result from tissue and muscle which does not recover and is not rehabilitated, or only recovers and is rehabilitated inadequately. These long-term effects can include urinary and faecal incontinence, but there is also the risk of orthopaedic problems, such as back pain.

The coil, the energy supply unit and the control unit are configured such that the coil can generate a magnetic field with an alternating pulse frequency, as stated further above.

Particularly in the use for pelvic floor muscle rehabilitation after childbirth, a gradual redevelopment of the muscles is provided. This affects the setting of the control unit to control the coil such that at the start of the treatment, a pulse frequency in the low range (2 Hz to 15 Hz) is selected. With a progressing recovery and rehabilitation of the pelvic floor muscles, the pulse frequency is adjusted to the medium range (20 Hz to 30 Hz) to increase the muscle volume, and it is adjusted to the high range (approximately 50 Hz up to the maximum contractility of the muscles) to increase the muscle power.

### Stimulation of Blood Flow

A further aspect of the invention provides a blood flow stimulating device which comprises a coil described above and in the following, an energy supply unit described above and in the following, and a control unit described above and in the following.

In this respect, the coil is configured such that it generates a magnetic field which stimulates the nerves in a muscle of a patient who is within the active region of the coil. The energy supply unit is configured to provide the energy supply for the coil, the control unit being arranged to control the energy supply of the coil by the energy supply unit.

In this respect, the energy supply is controlled by the control unit so that the magnetic fields generated by the coil are capable of stimulating the nerves in a muscle of a patient who is within the active region of the coil, so that due to the muscle contractions resulting therefrom, a flow of blood is stimulated through the tissue in the active region of the magnetic field.

The coil, the energy supply unit and the control unit can be configured as already described above.

The stimulation of blood flow in a patient is desired in various situations. In the case of patients who have to be in a prone position all day or practically all day, blood flow stimulation can be used to prevent decubitus (prevention of sores due to bedsores). Likewise, a blood flow stimulating device can be used for the prevention of thrombosis.

Furthermore, the blood flow stimulating device can be used to speed up healing in the case of swelling which occurs, for example, following dislocation, since an increased blood flow promotes healing.

A blood flow stimulating device can also be used to assist the dispersal of a medicament in the blood.

In particular, a blood flow stimulating device can also be used for diabetics who, according to their symptoms, are subject to an increased probability of a blood vessel blockage. A blood vessel blockage of this type can result in amputation of parts of the body.

In particular, the invention can exhibit its effect in a patient's body even through dressings, plaster casts and clothing.

The flow of blood through tissue is achieved in that a magnetic field produces muscle contractions which, in turn, entail a stimulation of the blood flow in the affected area of the body.

The stimulation of blood flow can require in particular a relatively long duration of an individual treatment session. It is also possible, for example, that a patient undergoes a long-lasting treatment.

The increased duration of the individual treatment session naturally means that the pulse frequency of the coil or of the magnetic field is set accordingly at a low level to prevent muscle fatigue. In this respect, the penetration depth of the magnetic field into the patient's body and the pulse frequency of the coil are to be calculated such that the muscle contractions correspond to a stress state which can be borne by the muscles in a long-term stress state.

Frequencies in the low range (2 Hz to 15 Hz) are appropriate for the pulse frequency.

An embodiment of the invention provides a couch for stimulating blood flow, which comprises a lying surface, a blood flow stimulating device according to an aspect of the invention, a guide device and an actuator.

The information provided above applies to the lying surface, to the guide device and to the actuator.

### Further Possible Uses

The invention can naturally also be used in other areas.

Thus, for example, every aspect and every embodiment of the invention can be used in conjunction with an office chair. In this embodiment, the invention can be used to build up muscle, to stimulate blood flow and to prevent everyday ailments. The term “everyday ailments” can be understood as meaning in particular those complaints which are caused by sitting frequently and for a long time, for example back pain and neck pain.

However, it is also possible to use the invention in conjunction with an armchair, for example.

Furthermore, the invention can be used for nerve stimulation in the case of paraplegia, to thus contribute, by the muscle contractions, to a strengthening and maintenance of the muscles. The invention can also be used for blood flow stimulation in paraplegia.

Furthermore, the invention can be used to stimulate the intestinal muscles. Particularly in the case of constipation caused by illness, old age or diet (slow digestive activity and/or obstruction), this can contribute to an improvement of the symptoms.

In addition, the invention can be used for the prevention of osteoporosis. Empirical investigations have shown that bones on which frequently moved muscles extend suffer significantly less often from osteoporosis than bones with muscles which are not moved frequently.

In particular, the invention can also be used to prevent osteoporosis, for example in patients who are wearing a plaster cast to heal a broken bone, since a magnetic field penetrates a cast.

Particularly suitable for use in the prevention of osteoporosis is a coil which is attached to a circular guide device, the guide device being moved along the bone to be treated. In this respect, the coil can be moved circularly along the guide device.

The invention has further possible diverse uses in sports medicine.

An operation can be avoided in the treatment of carpal tunnel syndrome in the hand joint. Carpal tunnel syndrome can result in weakness during a grasping action by the hand and can lead to a reduction in the hand's touch sensation.

Furthermore, the invention can be used to prevent osteoarthritis. The muscle contractions as a result of nerve stimulation caused by the magnetic field stimulate a greater secretion of fluid in the joints, as a result of which there is less friction of the bones in the joint.

The invention can also be used to artificially relax the muscles in preparation for a massage or for another orthopaedic treatment, thereby relieving any cramping of the muscle which may be present.

Accordingly, the invention can also be used to treat pain if, for example, a false posture is caused by muscle cramp. An artificial fatigue of the muscles, produced by the invention, contributes substantially to relieving the cramp and thereby to correcting the false posture.

Furthermore, it is possible to use the invention in the treatment of phantom pain following amputation.

The following table shows, by way of example, areas of use with exemplary settings of the control unit. Naturally, it should be noted that these values allow individual deviations subject to the characteristics of a person to be treated.

| | | On-time | Off-time | Treatment time | | Treatments |
|---|---|---|---|---|---|---|
| Diagnosis | Freq.(Hz) | (Sec.) | (Sec.) | (Minutes) | Intensity | per week |
| Stress incontinence | 23-50 | 5 | 5 | 20 | 100% | 3-5 |
| OAB | 2-50 | 5 | 5 | 20 | 100% | 3-5 |
| Mixed | 23-50-5 | 5 | 5 | 30 | 100% | 3-5 |
| incontinence | | | | | | |
| Faecal incontinence | 23-50 | 5 | 5 | 20 | 100% | 3-5 |
| Acute pain | | | | | | |
| (1^{st} cycle) | 25-35 | 3-5 | 3-5 | 5-7 | high (80%-100%) | daily |
| (2^{nd} cycle) | 45-50 | continuous | — | 5-7 | high (80%-100%) | daily |
| Chronic | 5-30 | continuous | — | 10-15 | high (80%-100%) | daily |
| pain | | | | | | |
| Erectile | 24 | 8 | 4 | 15-20 | 100% | 3-5 |
| dysfunction | 24 | 6 | 3 | 15-20 | 100% | 3-5 |
| Orgasm problems | 50 | 15 | 1 | 15-20 | 75-80% | daily |
| Cystitis | 40-50 | 5 | 5 | 15-20 | 45-55% | 3-5 |
| interstitialis | | | | | | |
| Incontinence | 10 | 5 | 5 | 10 | 50-100% | 3-5 |
| after radical | 50 | 5 | 5 | 10 | 50-100% | 3-5 |
| prostatectomy | | | | | | |
| Ejaculation force | 10 | 5 | 5 | 10 | 100% | 3-5 |
| | 50 | 5 | 5 | 10 | 100% | 3-5 |
| Chronic | 40-50 | 5 | 5 | 15-20 | 45-55% | 2-3 |
| prostatitis | | | | | | |
| Pelvic pain | 5-30 | continuous | — | 10-15 | 50-75% | daily |
| syndrome | | | | | | |
| Muscle volume | 23 | continuous | — | 10-2-10 | 100% | 3-5 |

The setting for the treatment of acute pain will be described here by way of example.

For the treatment in a first cycle, a pulse frequency of 25-35 Hz is set; here, as for all numerical information, this is naturally only a guidance value which can also be higher or lower, subject to the result of a diagnosis. The on-time, i.e. the time in which the pulse frequency energises the coil is approximately 3 to 5 seconds. A muscle located within the active region of the magnetic field of the coil is stimulated thereby during this time. The off-time is also 3 to 5 seconds; this time corresponds to the relaxation time of the muscle stimulated during the on-time. The entire treatment time amounts to 5 to 7 minutes, which is the duration of a treatment session. The coil is operated at high power, in this case at 80% to 100% of the power which the energy supply unit can deliver to the coil. The treatment is carried out daily.

In a second cycle, the pulse frequency of the coil is increased, for example to 45-50 Hertz and is applied continuously during the entire treatment session, i.e. the muscle is subjected to a continuous stress during the session and is not given any regeneration time. The remaining set values can remain the same, for example, but can also be adapted to a development of a patient's muscles during the course of the treatment. This means that the values can be increased or decreased.

The first and second cycles can be used on one day. However, they can also be used in a first treatment step and in a second treatment step, the first treatment step lasting several days and the second treatment step also lasting several days. In this respect, the treatment duration can be adjusted subject to progress made during the treatment and, for example, may be seven days for the first treatment step and 14 days for the second treatment step.

This list of areas of use of the invention is not considered to be conclusive. Instead, the invention can be widely used wherever it is necessary to produce a muscle contraction by the external application of nerve stimulation in a non-operative, non-invasive method.

In the following, embodiments of the invention will be described with reference to the figures.

### BRIEF DESCRIPTION OF THE FIGURES

FIG. 1 shows a coil, an energy supply unit and a control unit according to an embodiment of the invention.

FIG. 2 shows a coil having a coil core according to an embodiment of the invention.

FIG. 3 shows a further coil having a coil core according to an embodiment of the invention.

FIG. 4 shows a guide device with a coil according to an embodiment of the invention.

FIG. 5A shows a chair comprising an incontinence treatment device according to an embodiment of the invention.

FIG. 5B shows a wheelchair comprising an incontinence treatment device according to an embodiment of the invention.

FIG. 6 shows a hand-held unit according to an embodiment of the invention.

FIG. 7 shows a couch comprising guide devices and coils according to an embodiment of the invention.

FIG. 8 shows a bed comprising guide devices and coils according to an embodiment of the invention.

### DETAILED DESCRIPTION OF EMBODIMENTS

The illustrations in the figures are schematic and are not true to scale. When the same reference numerals are used in the following description of the figures, they relate to the same or similar elements.

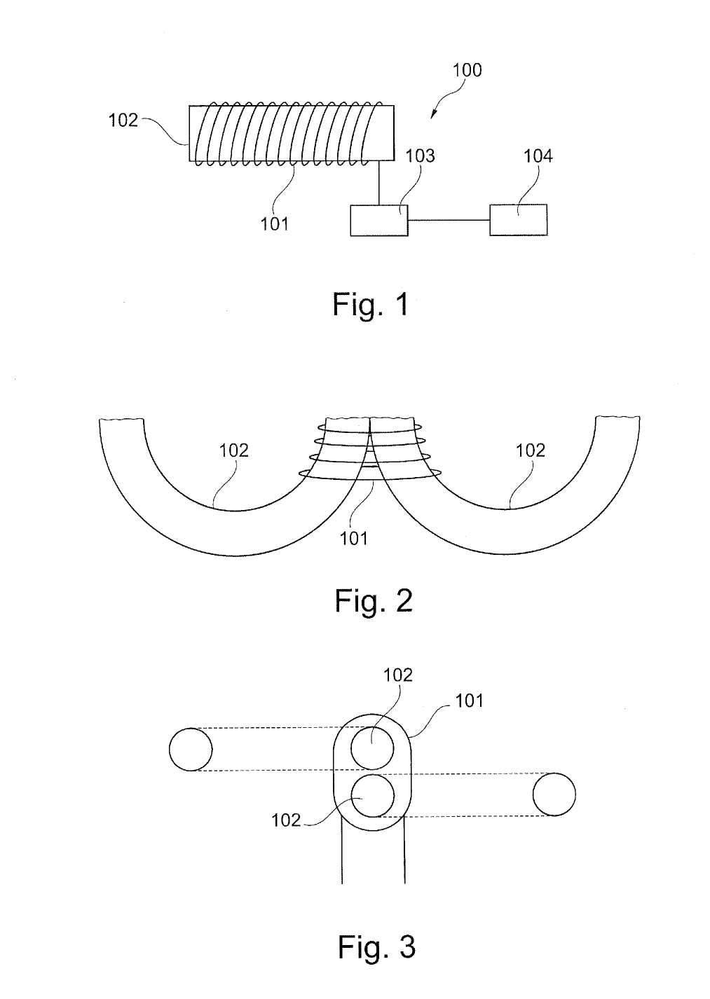

FIG. 1 shows an incontinence treatment device 100 according to an embodiment of the invention. The incontinence treatment device 100 comprises a coil 101, a coil core 102, an energy supply unit 103 and a control unit 104.

The coil 101 is connected to the energy supply unit 103 and to the control unit 104 so that the coil can be supplied with energy.

The coil 101 supplied thus with energy generates a magnetic field. The coil core 102 is arranged such that it focuses and directs the magnetic field generated by the coil 101.

The coil core 102 can be a magnetic or ferromagnetic item. The coil core 102 can have an oblong shape or any other shape, for example a semi-circular shape. However, other shapes are also possible for the configuration of the coil core 102.

The coil 101 has a plurality of windings around the coil core 102. The number of windings of the coil 101 is not restricted in the invention. In particular, the number, direction and spacing of the windings can be coordinated with the desired characteristics of the magnetic field generated by the coil 101 and the coil core 102. These characteristics include in particular the intensity of the magnetic field and the path thereof.

Of course, in addition to the shape of the coil core 102 and the construction of the coil 101, the energy supply unit and the control unit also influence the characteristics of the magnetic field generated by the coil 101. It is essentially the manual control of the energy supply unit by the control unit which predetermines the suitability for treatment of a disorder.

The incontinence treatment device shown in FIG. 1 can naturally be used for the treatment of other disorders, as described above and in the following, with an analogous construction and a different control of the energy supply unit by the control unit.

FIG. 2 shows the construction of a coil core 102 and of a coil 101 according to a further embodiment of the invention.

The double core 102 shown in FIG. 2 consists of two semicircular elements. However, the coil core elements 102 can also have a profile similar to an ellipse. Furthermore, the coil core elements 102 can have a different geometric structure, for example in the form of a rectangle where one edge of the rectangle is missing, thereby producing an opening at the coil core.

The coil 101 can jointly encompass the coil core elements 102, as shown in FIG. 2. In other words, this means that the windings of the coil 101 are guided around all the coil core elements 102. However, the windings of the coil 101 can also be configured such that they individually encompass the individual coil core elements 102.

In this respect, the windings which individually encompass the coil core elements 102 can be supplied jointly or individually with energy by the energy supply unit 103, the energy supply being controlled by the control unit 104.

The windings of the coil 101 can encompass any portion of the coil core 102. Thus, the windings of the coil 101 can extend over part of the coil core 102 and over the entire length of the coil core 102.

In a further embodiment of the invention, FIG. 3 shows an alternative arrangement of the coil cores 102 and of the coil 101. FIG. 3 is a plan view of an arrangement of two coil core elements 102 and of a coil 101 which is wound around them.

The coil 101 is arranged such that the windings thereof encompass two coil cores 102.

The first coil core 102 and the second coil core 102 have a transverse displacement relative to their respective longitudinal axis. Furthermore, the first coil core 102 and the second coil core 102 have been moved in their longitudinal direction such that they are next to one another at least to some extent.

The first coil core 102 and the second coil core 102 can have the same or different geometric dimensions. The coil 101 can jointly encompass the first coil core 102 and the second coil core 102, but the coil 101 can also encompass the first coil core 102 and the second coil core 102 separately.

Of course, it is possible for both a single coil core 102 and a plurality of coil cores 102 to be used in the course of the invention. The invention is not restricted to the use of one coil core or two coil cores. The coil cores which are used can be in any arrangement relative to one another.

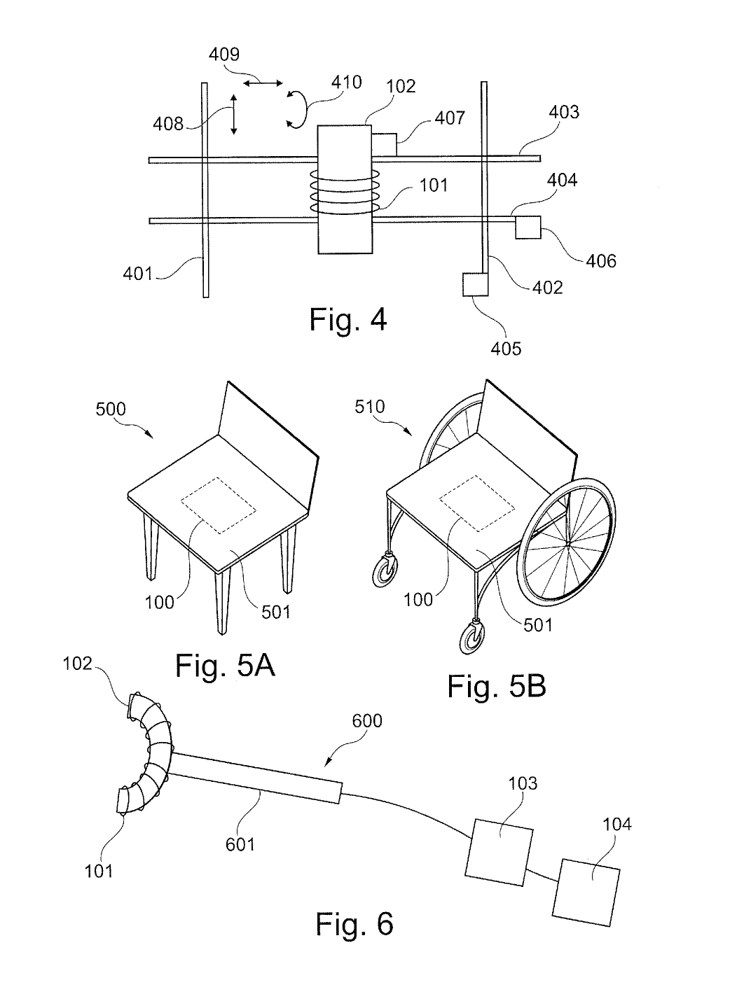

FIG. 4 shows a guide device with a coil 101 and a coil core 102 according to an embodiment of the invention.

The guide device comprises a first guide element 401, a second guide element 402, a third guide element 403 and a fourth third guide element 404. Furthermore, a first actuator 405, a second actuator 406 and a third actuator 407 are attached to the guide device.

The guide elements of the guide device can be, for example, guide rails. The actuators can be, for example, electric motors.

The coil 101 and the coil core 102 can be attached to a guide element 403. An actuator 407 can be attached to the guide element 403 and to the coil 101 or to the coil core 102 such that the actuator can bring about a change in position of the coil 101 and of the coil core 102 in the direction of arrow 410. The rotational direction in the direction of arrow 410 can be produced in particular in that the coil 101 and the coil core 102 rotate about a guide element 404 such that this guide element 404 forms the axis of rotation for the rotational movement in the direction of arrow 410.

An actuator 406 can be arranged such that the coil 101 and the coil core 102 are moved in the direction of arrow 409.

In this respect, the actuator 406 can cause the movement of the coil 101 and of the coil core 102 in particular via a chain revolving in the guide element 404 or via a revolving cable.

Particularly if the actuator 406 is an electric motor, a bidirectional movement in the direction of arrow 409 can be achieved by reversing the direction of rotation of the electric motor.

The actuator 405 is configured to bring about a movement of the coil 101 and of the coil core 102 (in the direction of arrow 408). In particular, the actuator 405 can be connected to the guide element 403 or to the guide element 404 by a chain or a cable in the guide element 402, so that the movement in the direction of arrow 408 by the coil 101 and by the coil core 102 is predetermined by a movement of guide element 403 and guide element 404.

Of course, the guide device can also comprise further guide elements. For example, the guide device can comprise further guide elements such that a movement of the coil 101 and of the coil core 102 is enabled in a direction which is vertical to the directions of movement along arrow 408 and arrow 409.

FIG. 5A shows an incontinence treatment chair 500 according to an embodiment of the invention. The incontinence treatment chair 500 has a seating surface 501 and an incontinence treatment device 100.

The incontinence treatment device 100 can be any exemplary embodiment of a coil having a coil core, an energy supply device and a control unit according to the invention. Thus, the chair 500 can be used not only for the treatment of incontinence, but it can also be used for any purpose of treatment described above and in the following, for example for pelvic floor muscle rehabilitation.

Of course, the position of the device 100 under the seating surface 501 can be changed. The device 100 can also be attached to the backrest of a chair, thereby making it possible for the back muscles to be treated.

The illustration of the chair is naturally only an example. The chair 500 can of course also be an office chair or an armchair, for example.

Of course, the chair 500 can also have a guide device, as described above and in the following, to change the position of the device 100.

FIG. 5B shows a wheelchair 510 comprising a seating surface 501 and an incontinence treatment device 100. In this case, the incontinence treatment device 100 is attached to the seating surface 501.

In particular, the wheelchair 510 can have a receiving device (not shown), which is configured such that the incontinence treatment device 100 can be attached in a modular manner to the wheelchair 510. This facilitates the treatment of wheelchair-dependent patients, because these patients do not have to get out of the wheelchair for treatment.

The wheelchair 510 shown by way of example can be any patient vehicle.

FIG. 6 shows a hand-held unit 600 for nerve stimulation by means of a magnetic field generated by the coil 100 via the coil core 102.

The coil 101 and the coil core 102 are attached to a handle portion 601 of the hand-held unit 600. The coil 101 is supplied with energy by the energy supply unit 103. The control unit 104 is configured to control the energy supply to the coil 101 by the energy supply unit 103.

The hand-held unit 600 can be used for all treatment purposes described above and in the following. In particular, it can be used to stimulate blood flow, to slow down muscle regression, for stroke rehabilitation and for the treatment of pain.

In particular, the hand-held unit 600 allows a flexible choice and a quick change-over of the part of a patient's body to be treated.

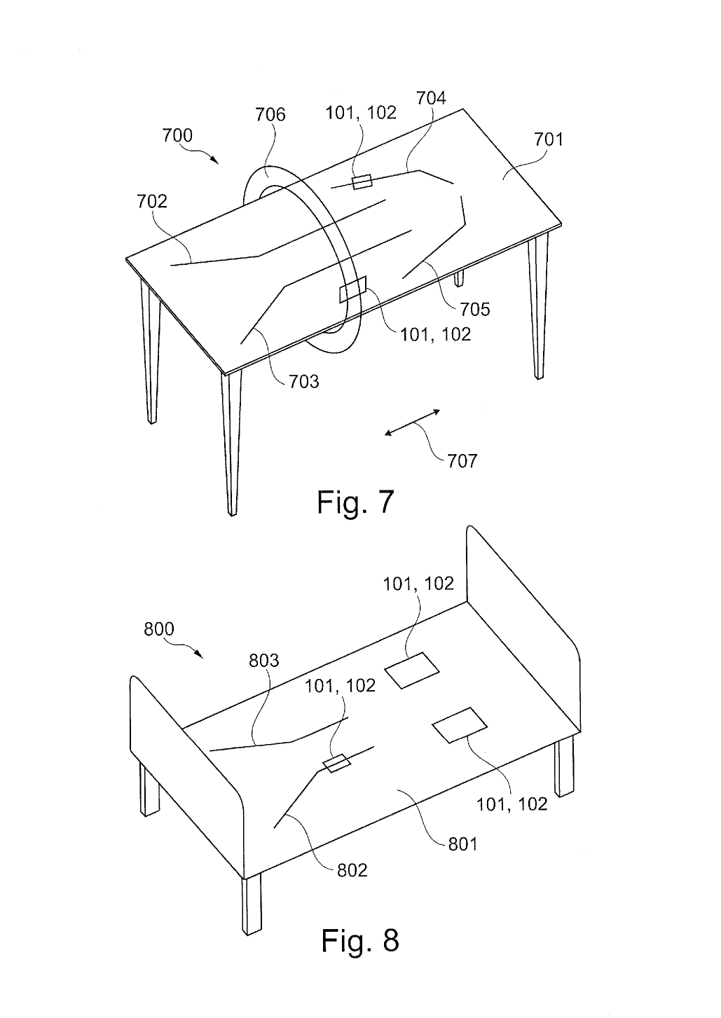

FIG. 7 shows a couch 700 comprising a lying surface 701 and a plurality of guide devices for a coil and a coil core according to an embodiment of the invention. The couch 700 has a first guide element 702, a second guide element 703, a third guide element 704 and a fourth guide element 705.

These guide elements can be adapted in particular to the path of the main muscle parts in the human body. In this respect, the back muscles, the leg muscles and the shoulder and arm muscles in particular can be simulated.

Furthermore, the couch 700 has a circular guide device 706 which encircles the lying surface 701. In this respect, the guide device 706 can move in the direction of arrow 707.

At least one coil 101 having a coil core 102 can be attached to each of the guide devices or guide elements mentioned above and in the following, the coil 101 and the coil core 102 being able to change their position along the path of the guide device or guide element. To change the position of the coil 101 and of the coil core 102, an actuator can be provided on the corresponding guide device.

The guide device 706 can be provided with one or more coils 101 and coil cores 102. Furthermore, the guide device 706 can be configured such that, when moving in the direction of arrow 707, it travels over the contour of the body of a patient who is lying on the lying surface 701. In particular, this can be performed such that a predetermined distance from the surface of the patient is observed at all times during the movement of the guide device 706 in the direction of arrow 707. In this respect, the coils 101 and coil cores 102 attached to the guide device 706 can naturally also be moved in a circular manner along the guide device 706. In particular, the coil 101 and the coil core 102 can circulate around a patient lying on the lying surface 701.

The rotational speed can be maintained constant or can be changed during the circular movement of the coil 101 and of the coil core 102 along the guide device 706. In particular, the rotational frequency of the coil 101 and of the coil core 102 on the guide device 706 can be adapted to the purpose of treatment.

The couch 700 can naturally also be, for example, a patient transport couch in an ambulance, the patient transport couch being equipped with guide devices, coils and coil cores.

A couch 700 as described above and in the following can be used for all treatment purposes described above and in the following. In particular, a couch 700 of this type can be used to stimulate blood flow, for stroke rehabilitation, to slow down muscle regression, for the treatment of pain and to accelerate muscle build-up.

FIG. 8 shows a bed 800 comprising a lying surface 801 and a first guide device 802 and a second guide device 803. At least one coil 101 having a coil core 102 as described above and in the following can be attached to the first guide device and to the second guide device. Furthermore, a plurality of coils 101 and coil cores 102 as described above and in the following can be attached to the lying surface 801.

In this respect, the first guide device 802 and the second guide device 803 can be formed in particular such that the path of the main muscles in the human body is simulated. At least one coil 101 having a coil core 102 can be attached in each case along the path of the first guide rail and of the second guide rail, so that the coil 101 and the coil core 102 can change their position.

Furthermore, a first coil 101 having a coil core 102 and a second coil 101 having a coil core 102 can be attached to the lying surface 801. These can be attached such that they are stationary.

The bed 800 can be in particular a hospital bed.

The coil 101 is a coil 101 as described above and in the following and the coil core 102 is a coil core 102 as described above and in the following.

Of course, the coil 101 is connected to an energy supply unit as described above and in the following and to a control unit as described above and in the following.

In this respect, the coil 101 and the coil core 102 can be used to treat all disorders described above and in the following. In particular, the bed 800 according to an embodiment of the invention can be used to stimulate blood flow in order to prevent decubitus (bedsores), for example. In the case of patients who need to be positioned, for example, this can influence the time intervals of the individual positioning intervals.

Here, it is again pointed out in particular that every embodiment of the invention can be used for every treatment purpose described above and in the following, since the treatment objective and the course of treatment are predetermined by the control of the energy supply by the control unit, the coil and the coil core merely serving to generate a corresponding magnetic field in order to carry out the desired treatment.

It is also pointed out that the embodiments in the figures described above do not restrict the number of guide devices and the number of coils. Each embodiment of the invention is capable of being provided with an indeterminate number of guide devices and coils as well as coil cores. Furthermore, the invention can also have in each embodiment a plurality of energy supply units and control units.

In addition, it is pointed out that the terms “comprising” and “having” do not exclude any other elements or steps and “a” or “one” does not exclude a plurality. It is also pointed out that features or steps which have been described with reference to one of the above embodiments can also be used combined with other features or steps of other embodiments described above. Reference numerals in the claims should not be construed as limiting the scope of the claims.

1-15. (canceled)

16. An incontinence treatment device, comprising:
a coil;
an energy supply unit; and
a control unit;
wherein the coil is configured to generate a magnetic field so that nerves are stimulated in a muscle of a patient located within an active region of the coil; the energy supply unit providing an energy supply for the coil; the control unit being arranged to control the energy supply to the coil by the energy supply unit; and the energy supply being controlled by the control unit so that the magnetic fields generated by the coil are capable of treating accompanying symptoms of incontinence.

17. The device according to claim 16, further comprising:
a guide device;
an actuator on the guide device for positioning the coil;
wherein the guide device is configured such that the actuator can change a position of the coil along the guide device; and the positioning of the coil being controlled by the control unit.

18. The device according to claim 16, comprising:
a seating surface;
wherein the incontinence treatment device is arranged under the seating surface.

19. A device for slowing down muscle regression, comprising:
a coil;
an energy supply unit; and
a control unit;
wherein the coil is configured to generate a magnetic field so that nerves are stimulated in a muscle of a patient located within an active region of the coil; the energy supply unit providing an energy supply for the coil; the control unit being arranged to control the energy supply to the coil by the energy supply unit; and the energy supply being controlled by the control unit so that the magnetic fields generated by the coil are capable of stimulating nerves in a muscle of a patient located within an active region of the coil so that, due to a resulting muscle activity, a muscle regression is slowed down.

20. The device of claim 19, wherein the device is configured as a hend held device.

21. The device of claim 19, wherein the device is configured as a couch and further comprising:
a lying surface;
a guide device; and
an actuator on the guide device for positioning the coil;
wherein the guide device is configured such that the actuator can change a position of the coil along the guide device; and the positioning of the coil being set by the control unit.

22. A body fat break-down device, comprising:
a coil;
an energy supply unit; and
a control unit;
wherein the coil is configured to generate a magnetic field so that nerves are stimulated in a muscle of a patient located within an active region of the coil; the energy supply unit providing an energy supply for the coil; the control unit being arranged to control the energy supply to the coil by the energy supply unit; and the energy supply being controlled by the control unit so that the magnetic fields generated by the coil are capable of stimulating nerves in a muscle of a patient located within an active region of the coil so that body fat is broken down.

23. The device of claim 16, wherein the energy supply being controlled by the control unit so that the magnetic fields generated by the coil are capable of stimulating nerves in a muscle of a patient located within an active region of the coil so that, due to the muscle contractions resulting therefrom, a build up of muscle is assisted.
